(19) United States
(12) Patent Application Publication
(10) Pub. No.: US 2022/0097018 A1
(43) Pub. Date: Mar. 31, 2022

(54) REACTION KETTLES, POLYPEPTIDE SYNTHESIS CLEAVAGE SYSTEM AND THEIR USE IN POLYPEPTIDE SYNTHESIS OR CLEAVAGE

(71) Applicant: CHANGZHOU SYNTHEALL PHARMACEUTICAL CO., LTD, Changzhou (CN)

(72) Inventors: Li-Qiang HOU, Changzhou (CN); Mei-Jun HU, Changzhou (CN); Dong-Hui YU, Changzhou (CN); Qi-Ge ZHANG, Changzhou (CN); Yin-Yin WANG, Changzhou (CN)

(21) Appl. No.: 17/489,040

(22) Filed: Sep. 29, 2021

(30) Foreign Application Priority Data
Sep. 30, 2020 (CN) 202011069392.9
Sep. 30, 2020 (CN) 202022216635.9

(51) Int. Cl. B01J 19/18

(52) U.S. Cl. CPC B01J 19/18

(57) ABSTRACT

The disclosure provides a reaction kettle, a polypeptide synthesis cleavage system and their use in polypeptide synthesis or cleavage. The reaction kettle comprises: (1) a kettle body; (2) a stirring device located at the upper part of the kettle body and extending to the interior of the kettle body; (3) a liquid feed port, a solid feed port, an inert gas inlet and an inert gas outlet located at the upper part of the kettle body; (4) a liquid discharge port and a liquid guiding groove at the bottom of the kettle body, wherein the liquid discharge port is located at the lowest point of the liquid guiding groove; (5) a filtering device located above the liquid guiding groove; (6) a solid discharge portpassing through the bottom of the kettle body and the filtering device; and (7) a discharge valve configured at the solid discharge port.

### TECHNICAL FIELD

The disclosure relates to a reaction kettle, which can separate solid and liquid discharge ports of the reaction kettle without disassembling the bottom of the reaction kettle for solid discharge, and can realize the inert gas protection in the whole process of the reaction. The disclosure also relates to a polypeptide synthesis and cleavage system and its use in polypeptide synthesis or cleavage.

### BACKGROUND

Solid-phase synthesis of polypeptides is one of the most important methods in the field of protein research and production.

The conventional solid-phase peptide synthesis method includes resin as an insoluble solid-phase carrier, wherein an amino acid with the amino group protected by a blocking group is first covalently linked to the solid-phase carrier. With the action of 20% piperidine/DMF, the protective group of the amino group is removed. Then the carboxyl group of the second amino acid with a blocked amino group is activated by N,N′-diisopropylcarbodiimide (DIC), and the second amino acid with the carboxyl group activated by DIC reacts with the amino group of the first amino acid linked to the solid-phase carrier to form a peptide bond, so that a dipeptide with a protective group is generated on the solid-phase carrier. Above peptide bond formation reaction is repeated to make the peptide chain grow from C-terminal to N-terminal until it reaches the required peptide chain length. Finally, the protective group is removed, the ester bond between the peptide chain and the solid carrier is cleaved with trifluoroacetic acid, and the side chain protective group is cleaved to obtain the synthesized crude peptide.

The traditional reaction kettle for polypeptide solid-phase synthesis has the following defects:

The solid and liquid ports of the reaction kettle are not separated, and the solid discharge is performed by disassembling the bottom of the reaction kettle, which is prone to material spillage loss and foreign matter introduction;

Failure to solve the problem of closed feeding of the reaction kettle, the feeding is practiced in an open way and the user needs to wear personal protective PPE. Air is easily mixed into the reaction kettle, which raises safety risk;

Failure to solve the problem of inert gas protection in the whole reaction process;

The existing software control systems and equipment hardware configurations fail to realize the air tightness inspection and monitoring of the reaction kettle;

Failure to achieve the automation of the whole production process, wherein human factors bring potential failure risk to production.

HAINAN JBPHARM CO. LTD has provided a polypeptide synthesizer for polypeptide solid-phase synthesis, but it has no inert gas protection mode. Its reaction kettle has poor air tightness, wherein the solvent is prone to volatilization, resulting in a production environment filled with foul odor. Air is easy to enter the reaction kettle, resulting in safety risk, and the solid discharge needs disassembling the bottom of the reaction kettle, the product is exposed for operation, and the odor of the solvent is heavy. The product is also prone to spillage loss and introduction of foreign matters.

Chinese patent CN205627928U discloses a multifunctional reaction kettle, which has a branch cylinder configured on the filtering device, and drives the filtering device to move upward by rotating the hand wheel of the upward discharge valve against the branch cylinder to realize solid-liquid separation. However, the solid and liquid in the reaction kettle are both discharged through the upward discharge valve, and the solid and liquid ports in the reaction kettle are not separated, which may lead to mixture and pollution, and the simultaneous separation of solid and liquid cannot be realized.

Obviously, there is still a need in the art to find a reaction kettle for polypeptide synthesis or cleavage, which can realize solid-liquid separation without manual disassembly of the bottom of the reaction kettle, and inert gas protection in the whole reaction process.

### SUMMARY

In order to solve the above technical problems, one aspect of the present disclosure provides a reaction kettle, which comprises:

(1) A kettle body;

(2) A stirring device located at the upper part of the kettle body and extending to the interior of the kettle body;

(3) A liquid feed port, a solid feed port, an inert gas inlet and an inert gas outlet located at the upper part of the kettle body;

(4) A liquid discharge port and a liquid guiding groove at the bottom of the kettle body, wherein the liquid discharge port is located at the lowest point of the liquid guiding groove;

(5) A filtering device located above the liquid guiding groove;

(6) A solid discharge port passing through the bottom of the kettle body and the filtering device; and

(7) A discharge valve configured at the solid discharge port.

Another aspect of the present disclosure provides a polypeptide synthesis and cleavage system, which comprises a synthesis device and a cleavage device, each independently including a reaction kettle of the present disclosure.

Another aspect of the present disclosure provides use of the reaction kettle of the present disclosure or the polypeptide synthesis and cleavage system of the present disclosure in polypeptide synthesis or cleavage.

### DESCRIPTION OF DRAWINGS

The disclosure is described in more detail below in combination with the attached drawings, wherein:

FIG. 1 is a front view of a reaction kettle according to an embodiment of the present disclosure;

FIG. 2 is a top view of the reaction kettle shown in FIG. 1;

FIG. 3 is a schematic diagram of the bottom of a reaction kettle according to an embodiment of the present disclosure;

FIG. 4 is a partial enlarged view of the bottom shown in FIG. 3;

FIG. 5 is a schematic diagram of a sieve plate of a reaction kettle according to an embodiment of the present disclosure;

FIG. 6 is a partially enlarged view of the sieve plate shown in FIG. 5;

FIG. 7 is a structural diagram of the bottom shown in FIG. 3 equipped with the sieve plate shown in FIG. 5;

FIG. 8 is a schematic diagram of a soft bag isolator of a reaction kettle according to an embodiment of the present disclosure;

FIG. 9 is a flow chart of synthesis steps in a polypeptide synthesis and cleavage system according to an embodiment of the present disclosure; and

FIG. 10 is a flow chart of cleavage steps in a polypeptide synthesis and cleavage system according to an embodiment of the present disclosure.

### EMBODIMENTS

The disclosure relates to a reaction kettle, which comprises a kettle body. In an embodiment of the present disclosure, the reaction kettle is closed, the reaction kettle body can be of any suitable pressure resistant material known in the art, and the cross-sectional shape of the reaction kettle body can be any suitable shape known in the art. In an embodiment of the present disclosure, the kettle body material is alloy steel, preferably stainless steel, and more preferably 316L stainless steel. In another embodiment of the present disclosure, the kettle body material is a corrosion-resistant material, such as glass lining, preferably a nickel base alloy, and more preferably a Hastelloy. In an embodiment of the present disclosure, the cross-sectional shape of the kettle body is circular.

The reaction kettle of the disclosure further comprises a stirring device located at the upper part of the kettle body and extending to the interior of the kettle body. The stirring device may be any suitable stirring device known in the art. In an embodiment of the present disclosure, the stirring device includes a motor and a stirring paddle.

The reaction kettle of the disclosure also comprises a liquid feed port, a solid feed port, an inert gas inlet and an inert gas outlet located at the upper part of the kettle body. In an embodiment of the present disclosure, the reaction kettle may comprise one or more liquid feed ports or solid feed ports. In an embodiment of the present disclosure, the solid feed port is configured with a soft bag isolator. The soft bag isolator may be any suitable soft bag isolator known in the art. The soft bag isolator may allow complete isolation of materials inside and outside the reaction system while feeding, and inert gas protection inside the soft bag isolator. In an embodiment of the present disclosure, the feed port of the soft bag isolator is connected to the solid feed port. As a non-limiting example, when in use, the solid material is placed in the soft bag isolator before feeding, the inert gas protection function runs, the opening of the soft bag isolator is closed, and the solid material is put into the feed port through gloves as needed. In an embodiment of the present disclosure, the inert gas inlet and the inert gas outlet are used for introducing and discharging the inert gas, respectively. In an embodiment of the present disclosure, the inert gas is nitrogen.

The reaction kettle of the present disclosure further comprises a liquid discharge port and a liquid guiding groove at the bottom of the kettle body, wherein the liquid discharge port is located at the lowest point of the liquid guiding groove. The shape of the liquid guiding groove can be any suitable shape known in the art as long as it can guide liquid to the liquid discharge port. In an embodiment of the present disclosure, the liquid discharge port is connected to a control valve or pump.

The reaction kettle of the present disclosure further comprises a filtering device located above the liquid guiding groove. In an embodiment of the present disclosure, the filtering device is a sieve plate. The sieve plate can be any suitable sieve plate known in the art, and the sieve plates with different mesh numbers can be selected according to the particle size of the solid particles to be isolated. In an embodiment of the present disclosure, the filtering device may include one or more sieve plates. In an embodiment of the present disclosure, the bottom of the kettle body is configured with a support plate to support the filtration device, and the liquid guiding groove is located between the support plates and/or between the support plate and the side wall of the kettle body. In an embodiment of the present disclosure, the lower surface of the filtering device and the bottom of the kettle body are configured with a fastener structure, so that the filtering device and the bottom of the kettle body can be fixed to each other through the fastener structure. In an embodiment of the present disclosure, the fastener structure is a fixture block. In an embodiment of the present disclosure, the edge of the filtering device is also configured with an annular sealing ring.

The reaction kettle of the present disclosure further comprises a solid discharge port passing through the bottom of the kettle body and the filtering device. In the present disclosure, the term “passing through the bottom of the kettle body and the filtering device” means that in the axial direction of the solid discharge port, the inlet of the solid discharge port is no lower than the upper surface of the filtering device, and the outlet of the solid discharge port is no higher than the lower surface of the bottom of the kettle body. In an embodiment of the present disclosure, the inlet of the solid discharge port is at the same height as the upper surface of the filtering device.

The reaction kettle of the present disclosure further comprises a discharge valve configured at the solid discharge port. The discharge valve may be any suitable valve known in the art. In an embodiment of the present disclosure, the discharge valve is an upward discharge valve. In another embodiment of the present disclosure, the discharge valve is a downward discharge valve. In an embodiment of the present disclosure, the discharge valve is connected to a filter press, which is configured with a soft bag isolator. In an embodiment of the present disclosure, the discharge valve is composed of a cylinder, a valve rod and a valve body, and the valve rod is controlled by the cylinder to drive the valve body to move up and down. The connection mode between the discharge valve and the solid discharge port may comprise threaded connection, flange connection, welding connection, clamp connection, cartridge connection or socket connection. In an embodiment of the present disclosure, the inlet of the discharge valve is at the same height as the upper surface of the filtering device.

In an embodiment of the present disclosure, the reaction kettle is configured with a pressure valve and a pressure gauge. The pressure valve and pressure gauge may be any suitable pressure valve and pressure gauge known in the art, respectively. In an embodiment of the present disclosure, the pressure valve is a PV valve and an XV valve, and the pressure gauge is a PT gauge. In an embodiment of the present disclosure, the inert gas inlet is connected to a gas feeding pipeline, the inert gas outlet is connected to a vent pipeline, and both the gas feeding pipeline and the vent pipeline are configured with PV valves and XV valves. In the present disclosure, the term “PV valve” is a pressure control valve used for controlling the pressure inside the reaction kettle. In the present disclosure, the term “XV valve” is an open-and-shut control valve, which has only open and shut states and is used to shut the valve.

The reaction kettle of the present disclosure may also be configured with a control system. The control system may be any suitable system known in the art capable of monitoring and controlling the production process. In an embodiment of the present disclosure, the control system is a Distributed Control System (DCS). In the present disclosure, the term “DCS” refers to a system used to monitor and control the production process in large-scale industrial production, also known as distributed control system. In an embodiment of the present disclosure, the reaction kettle is also configured with a pressure valve and a pressure gauge, and the control system is communicatively connected to the pressure valve and the pressure gauge. Accordingly, the control system can monitor the pressure inside the reaction kettle with the pressure gauge and control the feeding and discharge of inert gas with the pressure valve. In an embodiment of the present disclosure, the pressure inside the reaction kettle is maintained at 0.003-0.008 MPa through the control system. In an embodiment of the present disclosure, when the pressure inside the reaction kettle reaches a certain value and all valves are closed, the control system records the data of the pressure gauge in real time and calculates the leakage rate of the reaction kettle according to the change of the data over a period. Accordingly, when the calculated leakage rate of the reaction kettle exceeds a threshold, the control system can give an alarm. In an embodiment of the present disclosure, the kettle body is also configured with a tuning fork level gauge, and the control system is communicatively connected to the tuning fork level gauge, the liquid feed port and the liquid discharge port. Accordingly, when the reactant volume in the reaction kettle is excessive, the control system can monitor the reactant volume in the reaction kettle with the tuning fork level gauge and control the reactant volume in the reaction kettle by controlling the switches of the liquid feed port and the liquid discharge port. In an embodiment of the present disclosure, the kettle body is also configured with a temperature measuring port and a jacket, and the control system is communicatively connected to the temperature measuring port and the jacket. Accordingly, the control system can monitor the temperature inside the reaction kettle with the temperature measuring port and control the temperature in the reaction kettle by controlling the flowing of thermal medium in and from the jacket. In an embodiment of the present disclosure, the control system can collect the original data in the process of peptide synthesis and cleavage in real time.

The disclosure also relates to a polypeptide synthesis and cleavage system. The polypeptide synthesis and cleavage system comprises a synthesis device and a cleavage device, each independently including the reaction kettle described above in the present disclosure. In an embodiment of the present disclosure, the synthesis device comprises the reaction kettle described above in the present disclosure, and its discharge valve is connected to a filter press. In an embodiment of the present disclosure, the solids in the filter press are manually or automatically transferred to a drying device, and the solids in the drying device are manually or automatically transferred to the solid feed port of the cleavage device. In an embodiment of the present disclosure, the filter press and/or drying device may be configured with a soft bag isolator for transferring solids. In another embodiment of the present disclosure, the filter press may be connected to a drying device, and the drying device may be connected to a solid feed port of the cleavage device. The filter press and drying device may be any suitable filter press and drying device known in the art, respectively. In an embodiment of the present disclosure, the drying device may be a flat oven. In an embodiment of the present disclosure, the cleavage device includes the reaction kettle described above in the present disclosure. In an embodiment of the present disclosure, the polypeptide synthesis and cleavage system comprise a control system, and the control system can be communicatively connected to a pump or valve in the polypeptide synthesis and cleavage system to control the material transportation inside or between the synthesis device and the cleavage device. In an embodiment of the present disclosure, the material may comprise an inert gas, a reaction reagent and an intermediate.

The disclosure also relates to use of the reaction kettle described above in the present disclosure or the polypeptide synthesis and cleavage system described above in the present disclosure in polypeptide synthesis or cleavage.

Beneficial Effect

1) Compared with conventional reaction kettles, the reaction kettle of the present disclosure allows the separation of solid and liquid ports without disassembling the bottom of the reaction kettle, closed solid discharge by connecting the filter press to the solid discharge port of the reaction kettle, effective protection of the health of operators, reduced environmental pollution, reduced product loss or introduction of foreign matters;

2) Compared with the conventional reaction kettles, the reaction kettle of the present disclosure allows enclosed feeding into the reaction kettle, thereby preventing dust diffusion and reducing environmental pollution;

3) Compared with the conventional reaction kettles, the reaction kettle of the present disclosure allows inert gas protection of the whole reaction process to effectively prevent the potential safety risk caused by the incorporation of air into the reaction kettle;

4) Compared with the conventional reaction kettle, the reaction kettle of the present disclosure allows programmed inspection for the air tightness of the equipment, which can effectively avoid misjudgment in the inspection, so as to ensure the success of the production process;

5) Compared with the conventional reaction kettle, the reaction kettle of the present disclosure allows programmed design of the whole production process to make the whole process follow the established process and reduce the probability of human error in the production process; and

6) Compared with the conventional reaction kettle, the reaction kettle of the present disclosure allows high automatization of the production process with the control system, which provides a guarantee for the commercial production of products.

The technical solution of the present disclosure will be clearly and completely described below in combination with the attached drawings. Obviously, the examples are only part of the embodiments of the present disclosure, not all of them. Based on the examples of the present disclosure, all other examples obtained by those skilled in the art without creative works fall into the protection scope of the present disclosure.

### EXAMPLE

### Example 1

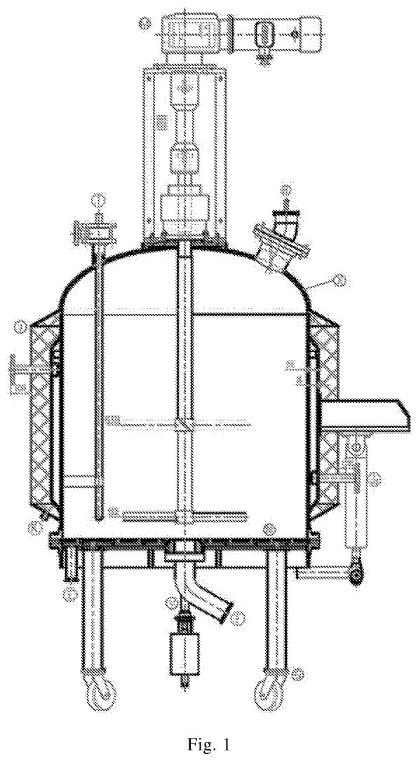

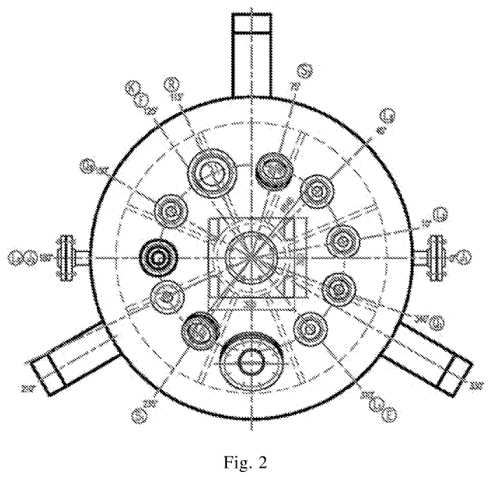

A reaction kettle, of which the structure is shown in FIGS. 1-2, comprising: kettle body X, stirring device M, liquid feed port L1, liquid feed port L2, liquid feed port L3, tuning fork port Ls, inert gas inlet R, inert gas outlet (not shown in the figure), spray port Q1, spray port Q2, solid feed port H, temperature measurement port T, mirror port S1, mirror port S2, sieve plate N, liquid discharge port E, upward discharge valve V, jacket J, and signal port K; wherein the stirring device M is configured to stir the materials in the reaction kettle; the liquid feed port L1, liquid feed port L2, liquid feed port L3, spray port Q1 and spray port Q2 are configured for liquid reactant feeding; the tuning fork port LS is configured for inserting the tuning fork level gauge; the inert gas inlet/outlet R is configured to feed and discharge inert gas; the solid feed port H is configured for solid reactant(resin) feeding; the temperature measuring port T is configured for inserting a thermometer; the mirror port S1 and the mirror port S2 are configured for observing the reaction in the kettle body X in real time; the sieve plate n is configured for filtering and separating the solid and liquid in the kettle body X; the liquid discharge port E is configured to discharge the liquid in the kettle body X; the upward discharge valve V is configured with upward discharge valve discharge port F for solid discharge; the upper and lower parts of the jacket J are respectively configured with jacket inlet J1 and jacket outlet J2 for feeding and discharge the heating medium; and the signal port K is configured for communication connection with DCS system (not shown in the figure) to monitor and control the reaction in the kettle body X.

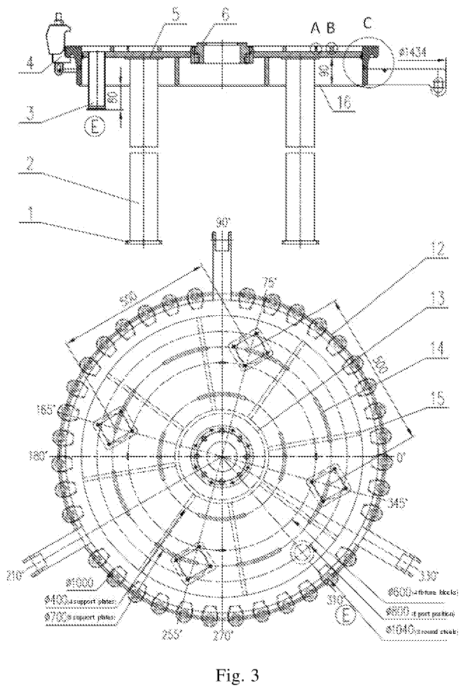

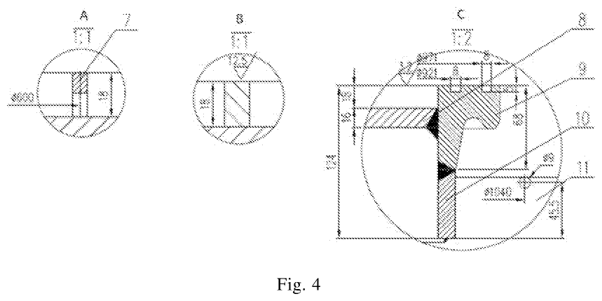

FIG. 3 shows the bottom of the reaction kettle (without sieve plate), and FIG. 4 shows the partial enlarged views of portions A, B and C, comprising: caster connecting plate 1, connecting pipe 2, quick fitting joint 3, clip 4, backing plate 5, upward discharge valve flange 6, lower clamp 7, bottom plate 8, container flange 9, cylinder 10, chassis connecting rod 11, round steel 12, short connection 13, support plate 14, square plate 15 and sealing plate 16.

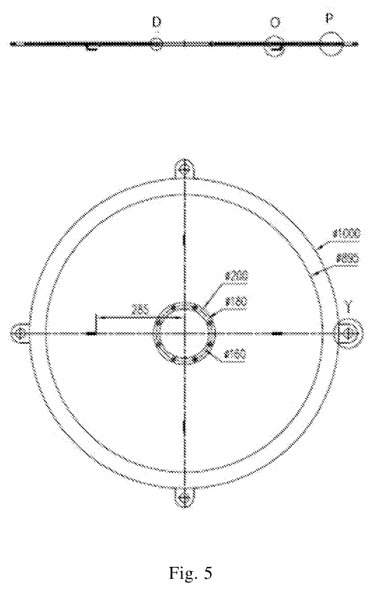

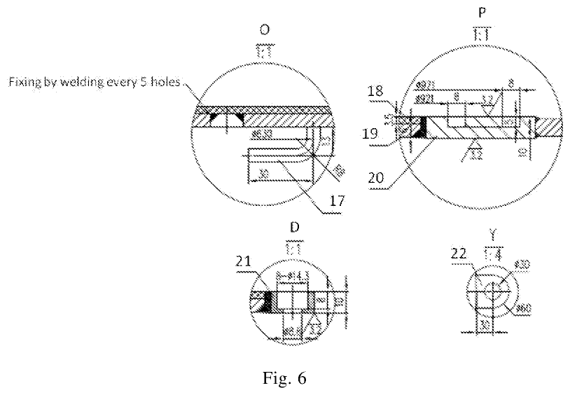

FIG. 5 shows a schematic diagram of the sieve plate of the reaction kettle, and FIG. 6 shows a partial enlarged view of portions D, O, P and Y, comprising: round steel 17, sintering plate 18, filter plate 19, ring 20, ring 21 and ear plate 22.

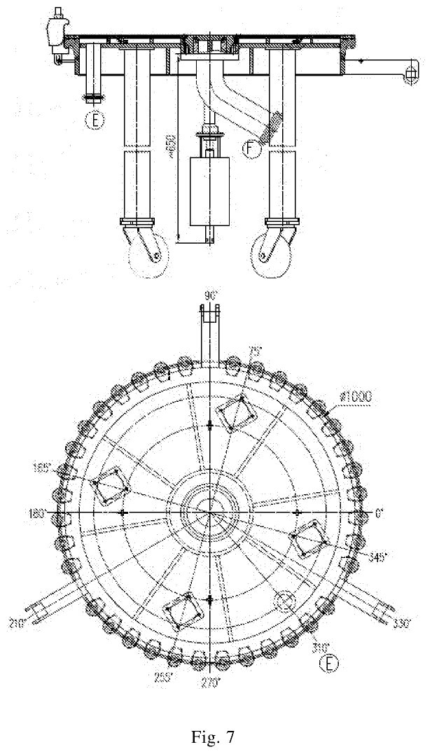

FIG. 7 shows a schematic diagram of the bottom equipped with a sieve plate, wherein the round steel 17 is fastened with the lower clamp 7, so that the sieve plate is fixed on the bottom.

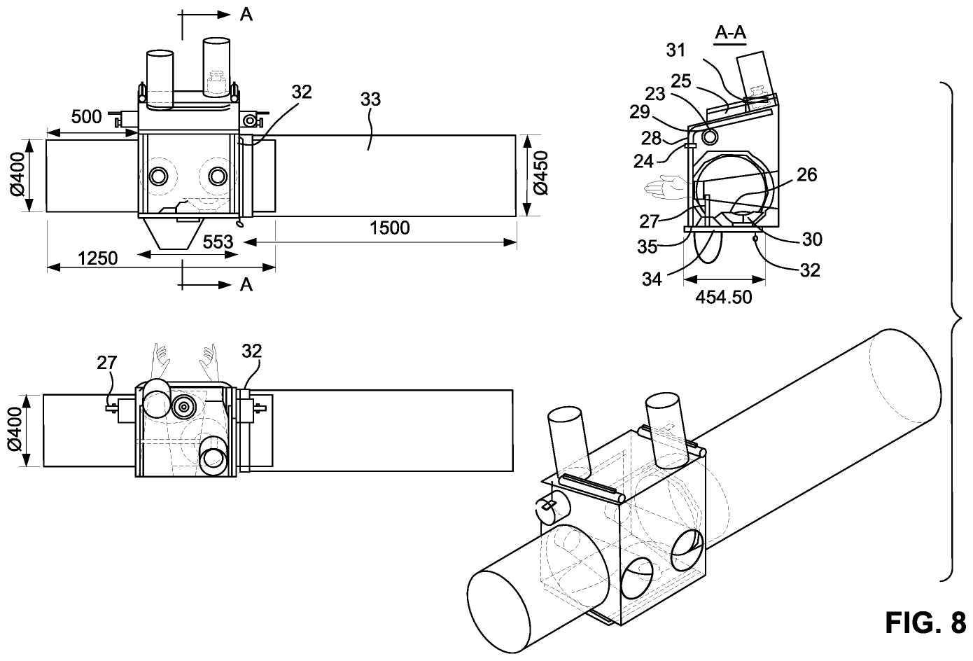

FIG. 8 shows the schematic diagram of the soft bag isolator of the reaction kettle, comprising: tray weldment 23, soft bag support weldment 24, intermediate pull rod weldment 25, soft bag weldment 26, cover plate 27, ball valve 28, filter element support 29, sintering screen plate filter device 30, funnel weldment 31, sampling assembly 32, feed barrel assembly 33, PE bag 34 and hoop 35.

### Example 2

A polypeptide synthesis and cleavage system, which comprises a synthesis device and a cleavage device.

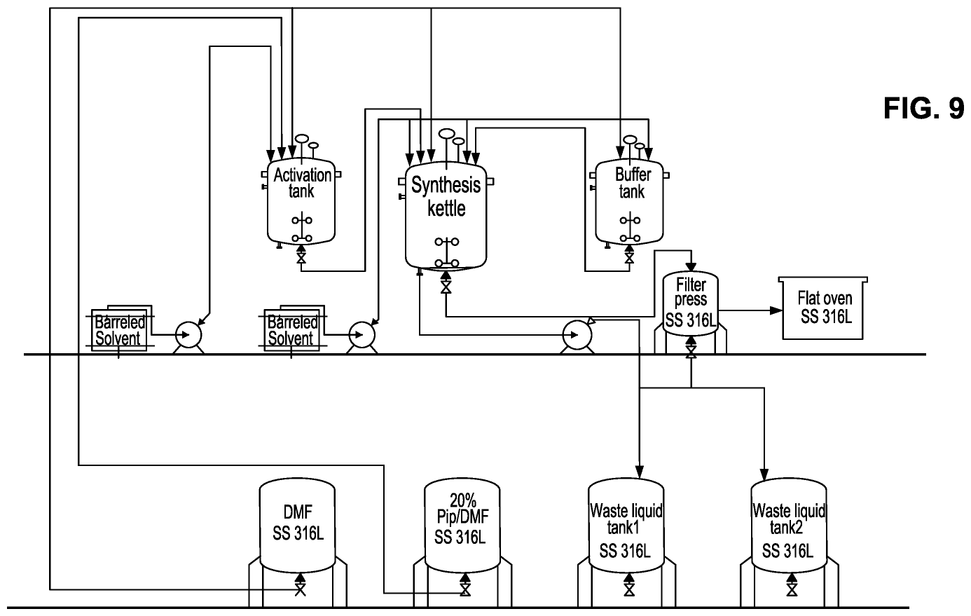

As shown in FIG. 9, the synthesis device comprises a barreled solvent container 1, a barreled solvent container 2, a DMF reservoir, a 20% piperidine/DMF reservoir, an activation tank, a synthesis kettle, a buffer tank, a waste liquid tank 1, a waste liquid tank 2, a filter press and a flat oven; wherein the synthesis kettle is a reaction kettle as described in Example 1; the barreled solvent container 1 is connected to the feed inlet of the activation tank through a pump; the DMF reservoir and the 20% piperidine/DMF reservoir are connected to the feed port of the activation tank; the barreled solvent container 2 is connected to the feed port of the buffer tank through a pump; the DMF reservoir is connected to the feed port of the buffer tank; the barreled solvent container 2 is connected to a liquid feed port or spray port of the synthesis kettle through a pump; the discharge port of the activation tank, the discharge port of the buffer tank and the DMF reservoir are connected to a liquid feed port or spray port of the synthesis kettle; the liquid discharge port of the synthesis kettle is connected to the waste liquid tank 1 through a pump; the discharge port of the upward discharge valve of the synthesis kettle is connected to the filter press; the liquid discharge port of the filter press is connected to the waste liquid tank 1 and the waste liquid tank 2; and solids are transferred from the solid discharge port of the filter press to the flat oven.

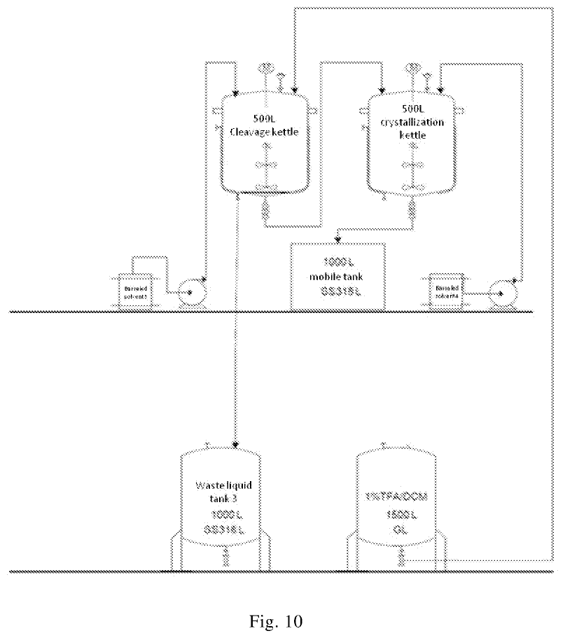

As shown in FIG. 10, the cleavage device comprises a barreled solvent container 3, a barreled solvent container 4, a cleavage kettle, a crystallization kettle, a mobile tank, a waste liquid tank 3 and a 1% TFA/DCM reservoir; wherein the cleavage kettle is a reaction kettle as described in Example 1; the barreled solvent container 3 is connected to a liquid feed port or spray port of the cleavage kettle through a pump; the 1% TFA/DCM reservoir is connected to a liquid feed port or spray port of the cleavage kettle; the liquid discharge port of the cleavage kettle is connected to the waste liquid tank 3; the barreled solvent container 4 is connected to the feed port of the crystallization kettle through a pump; the discharge port of the upward discharge valve of the cleavage kettle is connected to the feed port of the crystallization kettle; and the discharge port of the crystallization kettle is connected to the mobile tank.

### Example 3

Experiment of Polypeptide Synthesis and Cleavage

(1) Equipments (The Filtration Accuracy of Sieve Plate in the Synthesis Kettle and Cleavage Kettle is 40 μm):

| Name | Specifications | Material | Manufactor |
|---|---|---|---|
| Synthesis kettle | 500 L | SS316L | Zhejiang Chengxin |
| Activation tank | 300 L | SS316L | Zhejiang Chengxin |
| Buffer tank | 300 L | SS316L | Zhejiang Chengxin |
| Filter press | 100 L | SS316L | Shanghai Sanjian |
| Flat oven | 32disc | SS316L | Nanjing Yangtze |
| | | | River |
| Cleavage kettle | 500 L | HC & GL | Zhejiang Chengxin |
| Crystallization kettle | 500 L | GL | Zhejiang Chengxin |
| Mobile tank | 1000 L | SS316L | Zhejiang Chengxin |

The equipments in the above table are connected as described in Example 2 to form a polypeptide synthesis and cleavage system as described in Example 2.

(2) Raw Materials:

| Name | Manufactor | Level |
|---|---|---|
| Rink amide linker | Xi'an Lanxiao | Test grade |
| resin (MBHA) | | |
| Fmoc-Ala-OH | Zhengyuan Shenghua | Test grade |
| Fmoc-Arg-OH | Zhengyuan Shenghua | Test grade |
| Fmoc-Asn-OH | Zhengyuan Shenghua | Test grade |
| Fmoc-Asp-OH | Zhengyuan Shenghua | Test grade |
| Fmoc-Cys-OH | Zhengyuan Shenghua | Test grade |
| Fmoc-Gln-OH | Zhengyuan Shenghua | Test grade |
| Fmoc-Glu-OH | Zhengyuan Shenghua | Test grade |
| Fmoc-Gly-OH | Zhengyuan Shenghua | Test grade |
| Fmoc-His-OH | Zhengyuan Shenghua | Test grade |
| Fmoc-Ile-OH | Zhengyuan Shenghua | Test grade |
| Fmoc-Leu-OH | Zhengyuan Shenghua | Test grade |
| Fmoc-Lys-OH | Zhengyuan Shenghua | Test grade |
| Fmoc-Met-OH | Zhengyuan Shenghua | Test grade |
| Fmoc-Phe-OH | Zhengyuan Shenghua | Test grade |
| Fmoc-Pro-OH | Zhengyuan Shenghua | Test grade |
| Fmoc-Ser-OH | Zhengyuan Shenghua | Test grade |
| Fmoc-Thr-OH | Zhengyuan Shenghua | Test grade |
| Fmoc-Trp-OH | Zhengyuan Shenghua | Test grade |
| Fmoc-Tyr-OH | Zhengyuan Shenghua | Test grade |
| Fmoc-Val-OH | Zhengyuan Shenghua | Test grade |
| Oxyma | Suzhou Haofan | Test grade |
| DIPEA | Changzhou Tianhua | Test grade |
| DIC | Suzhou Haofan | Test grade |
| Piperidine | Vertellus Specilties Inc | Test grade |
| methanol | Shanghai Huayi | Test grade |
| DMF | Shandong Hualu | Test grade |
| DCM | Ningbo Juhua | Test grade |
| IPA | Kailing chemical | Test grade |
| MTBE | Qilu Petrochemical | Test grade |
| acetic anhydride | Celanese | Test grade |
| NMM | Liyang Yutian | Test grade |
| TFA | Wujiang Chuangwei | Test grade |
| | Chemical Co., Ltd | |
| nitrogen | Changzhou Jinghua | Test grade |
| | Industrial Gas Co., Ltd | |

(3) Experimental Method

a. Inerting and Nitrogen Protection

Nitrogen protection was provided by injecting nitrogen into the activation tank, synthesis kettle and buffer tank through the inert gas inlet, and monitoring and controlling the pressure within the activation tank, synthesis kettle and buffer tank to maintain at normal level through DCS system.

b. Resin Swelling

10 kg MBHA resin was fed into the synthesis kettle through the solid feed port, 150 L DMF was added into the synthesis kettle through a liquid feed port or spray port from the DMF reservoir. The stirring device was activated, and the resin was swollen at 20° C. for 25 minutes, and the liquid in the synthesis kettle was discharged to the waste liquid tank 1 through the liquid discharge port after the resin swelled. This step was repeated one time.

c. Deprotection

150 L 20% piperidine/DMF was added into the buffer tank through the feed port, then entered the synthesis kettle through the discharge port of the buffer tank and a liquid feed port or spray port of the synthesis kettle. The stirring device was activated and the material was stirred at 20° C. for 25 minutes. The liquid in the synthesis kettle was discharged to the waste liquid tank 1 through the liquid discharge port after the reaction was complete.

d. Washing

50 L DMF was added into the buffer tank from the DMF reservoir through the feed port, and then entered the synthesis kettle through the discharge port of the buffer tank and a liquid feed port of the synthesis kettle. This step was repeated for three times.

e. Resin Washing After Deprotection

The stirring device was activated and the material was stirred at 20° C. for 25 minutes. After washing, the liquid in the synthesis kettle was discharged to the waste liquid tank 1 through the liquid outlet. Then, 150 L DMF was added into the synthesis kettle through a liquid feed port or spray port from the DMF reservoir. The stirring device was activated and the material was stirred at 20° C. for 25 minutes, and then the liquid in the synthesis kettle was discharged to the waste liquid tank 1 through the liquid discharge port. This step was repeated for three times.

f. Amino Acid Activation

Amino acids were added to the activation tank through the feed port. 100 L DMF was added to the activation tank from the DMF reservoir through the feed port. Amino acids, Oxyma and DIC were added through the solid feed port. The stirring device was activated, the material was stirred at 20° C. for 25 minutes, and then entered the synthesis kettle through the discharge port of the activation tank and a liquid feed port of the synthesis kettle.

g. Reaction

The stirring device was activated and the material was stirred at 20° C. for 2 hours.

h. Sampling and Detection

Stirring was stopped and samples were taken.

i. Washing

50 L DMF is added to the activation tank from the DMF reservoir through the feed port, and then entered the synthesis kettle through the discharge port of the activation tank and a liquid feed port or spray port of the synthesis kettle. This step was repeated for three times.

j. Resin Washing

The stirring device was activated and the material was stirred at 20° C. for 25 minutes. After washing, the liquid in the synthesis kettle was discharged to the waste liquid tank 1 through the liquid discharge port. 150 L DMF was added into the synthesis kettle through a liquid feed port or spray port from the DMF reservoir. The stirring device was activated, and then the liquid in the synthesis kettle was discharged to the waste liquid tank 1 through the liquid discharge port. This step was repeated for three times.

k. End Blocking

120 L DMF, 15 L acetic anhydride and 15 L NMM were added to the buffer tank from the DMF reservoir through the feed port. The stirring device was activated, and the material was stirred for 3 minutes, then entered the synthesis kettle through the discharge port of the buffer tank and a liquid feed port or spray port of the synthesis kettle. The stirring device was activated, the material was stirred at 20° C. for 25 minutes, and then the liquid in the synthesis kettle was discharged to the waste liquid tank 1 through the liquid discharge port.

l. Washing

50 L DMF was added to the buffer tank from the DMF reservoir through the feed port, and then entered the synthesis kettle through the discharge port of the buffer tank and a liquid feed port or spray port of the synthesis kettle. This step was for three times.

m. DMF Washing

The stirring device was activated, the material was stirred at 20° C. for 25 minutes. After washing, the liquid in the synthesis kettle was discharged to waste liquid tank 1 through the liquid discharge port. 150 L DMF was added into the synthesis kettle through a liquid feed port or spray port from the DMF reservoir. The stirring device was activated, and then the liquid in the synthesis kettle was discharged to the waste liquid tank 1 through the liquid discharge port. This step was repeated for three times.

Then, steps C-M were repeated, wherein different amino acids to be connected were added each time based on the amino acid sequence of the desired polypeptide until all amino acids of the whole peptide chain of the polypeptide were connected.

n. DCM Washing

DCM was added into the synthesis kettle through a liquid feed port or spray port. The stirring device was activated, and then the liquid in the synthesis kettle was discharged to the waste liquid tank 1 through the liquid discharge port. This step was repeated twice.

o. Methanol Washing

Methanol was added into the synthesis kettle through a liquid feed port or the spray port. The stirring device was activated, and then the liquid in the synthesis kettle was discharged to the waste liquid tank 1 through the liquid discharge port. This step was repeated twice.

p. Discharge from Synthesis Kettle

Methanol was added into the synthesis kettle through a liquid feed port or spray port. The stirring device, the upward discharge valve and the pressure valve were opened to feed nitrogen to increase the pressure of the synthesis kettle. The liquid in the synthesis kettle was discharged to the waste liquid tank 1 through the liquid discharge port, and the solid in the synthesis kettle was discharged to the filter press through the solid discharge port. Then stirring was stopped, the pressure valve was closed and the upward discharge valve was opened.

q. Drying

The solid was dried in the filter press, the liquid in the filter press was discharged to the waste liquid tank 2 through the discharge port, and then the dried solid was placed in the flat oven for drying.

r. DCM Swelling Washing Resin

The resin was fed into the cleavage kettle through the solid feed port, and DCM was added into the cleavage kettle through a liquid feed port or spray port. The stirring device was activated, and the liquid in the cleavage kettle was discharged to the waste liquid tank 3 through the liquid discharge port after the resin was swollen.

s. Soft Cleavage

1% TFA/DCM was added into the cleavage kettle from 1% TFA/DCM reservoir. The stirring device was activated, and the cleavage liquid entered the crystallization kettle through the liquid outlet of the cleavage kettle and the feed inlet of the crystallization kettle.

t. Neutralization

1 L pyridine was added into the crystallization kettle through a liquid feed port or spray port. The stirring device was activated, the material was stirred at 20° C. for 25 minutes, and the liquid in the crystallization kettle was discharged to the mobile tank through the liquid discharge port. Then, DCM was added to the cleavage kettle through a liquid feed port or spray port. The stirring device, the upward discharge valve and the pressure valve were opened to feed nitrogen to increase the pressure of the synthesis kettle, and the solid liquid suspension in the cleavage kettle was discharged to the filter press (not shown) through the solid discharge port.

The above is only a preferred embodiment of the invention and does not limit the invention in any way. It should be noted that those skilled in the art can make several improvements and supplements without departing from the method of the invention, which should also be regarded as the protection scope of the invention. Those skilled in the art, without departing from the spirit and scope of the invention, can make some changes, modifications, and equivalent changes by using the technical contents disclosed above, which are equivalent embodiments of the invention. Meanwhile, any equivalent changes, modifications, and evolution of the above embodiments according to the essential technology of the invention still fall within the scope of the technical solution of the invention.

1. A reaction kettle comprising:
(1) A kettle body;
(2) A stirring device located at the upper part of the kettle body and extending to the interior of the kettle body;
(3) A liquid feed port, a solid feed port, an inert gas inlet and an inert gas outlet located at the upper part of the kettle body;
(4) A liquid discharge port and a liquid guiding groove at the bottom of the kettle body, wherein the liquid discharge port is located at the lowest point of the liquid guiding groove;
(5) A filtering device located above the liquid guiding groove;
(6) A solid discharge port passing through the bottom of the kettle body and the filtering device; and
(7) A discharge valve configured at the solid discharge port.

2. The reaction kettle according to claim 1, wherein the discharge valve is an upward discharge valve, and the filtering device is of a sieve plate structure.

3. The reaction kettle according to claim 1, wherein the solid feed port is configured with a soft bag isolator.

4. The reaction kettle according to claim 1, wherein the discharge valve is connected to a filter press, and the filter press is configured with a soft bag isolator.

5. The reaction kettle according to claim 1, wherein the reaction kettle is also configured with a pressure valve and a pressure gauge.

6. The reaction kettle according to claim 1, wherein the reaction kettle is also configured with a control system.

7. The reaction kettle according to claim 6, wherein the reaction kettle is further configured with a pressure valve and a pressure gauge, and the control system is communicatively connected to the pressure valve and the pressure gauge.

8. The reaction kettle according to claim 6, wherein the kettle body is further configured with a tuning fork liquid level gauge, and the control system is communicatively connected to the tuning fork liquid level gauge, the liquid feed port, and the liquid discharge port.

9. A polypeptide synthesis and cleavage system, wherein the polypeptide synthesis and cleavage system comprises a synthesis device and a cleavage device, each independently comprising a reaction kettle according to claim 1.

10. Use of the reaction kettle according to claim 1 in polypeptide synthesis or cleavage.

11. Use of the polypeptide synthesis and cleavage system according to claim 9 in polypeptide synthesis or cleavage.
